(19) United States
(12) Patent Application Publication
(10) Pub. No.: US 2024/0065572 A1
(43) Pub. Date: Feb. 29, 2024

(54) System and Method for Tracking an Object Based on Skin Images

(71) Applicants: Carnegie Mellon University, Pittsburgh, PA (US); University of Pittsburgh - Of the Commonwealth System of Higher Education, Pittsburgh, PA (US)

(72) Inventors: John Michael Galeotti, Pittsburgh, PA (US); George DeWitt Stetten, Pittsburgh, PA (US); Chun-Yin Huang, Pittsburgh, PA (US)

(21) Appl. No.: 18/280,283

(22) PCT Filed: Mar. 4, 2022
(86) PCT No.: PCT/US22/18835

§ 371 Date: Sep. 5, 2023

Related U.S. Application Data
(60) Provisional application No. 63/156,521, filed on Mar. 4, 2021.

(51) Int. Cl. A61B 5/06; A61B 5/00; G06T 7/246; G06T 7/73

(52) U.S. Cl. CPC A61B 5/061; A61B 5/0077; A61B 5/742; G06T 7/246; G06T 7/75; G06T 2207/10132; G06T 2207/20016; G06T 2207/30088

(57) ABSTRACT

Provided is a system, method, and computer program product for tracking an object based on skin images. A method includes capturing, with at least one computing device, a sequence of images with a stationary or movable camera unit arranged in a room, the sequence of images including the subject and an object moving relative to the subject, and determining, with at least one computing device, the pose of the object with respect to the subject in at least one image of the sequence of images based on computing or using a prior surface model of the subject, a surface model of the object, and an optical model of the stationary or movable camera unit.

### CROSS REFERENCE TO RELATED APPLICATION

This application claims priority to U.S. Provisional Patent Application No. 63/156,521, filed Mar. 4, 2021, the disclosure of which is incorporated herein by reference in its entirety.

### GOVERNMENT LICENSE RIGHTS

This invention was made with Government support under 1R01EY021641 awarded by the National Institute of Health, and W81XWH-14-1-0370 and W81XWH-14-1-0371 awarded by the Department of Defense. The Government has certain rights in the invention.

### BACKGROUND

### 1. Field

This disclosure relates generally to tracking the motion of one or more objects relative to visible skin on a subject by means of computer vision from a freely movable camera and, in non-limiting embodiments, to systems and methods for tracking an ultrasound probe relative to a subject's skin and body and, in other non-limiting embodiments, to systems and methods for tracking a head-mounted display by means of cameras viewing a subject.

### 2. Technical Considerations

Ultrasound is a widely used clinical imaging modality for monitoring anatomical and physiological characteristics. Ultrasound combines several advantages including low-cost, real-time operation, a small size that is easy to use and transport, and a lack of ionizing radiation. These properties make ultrasound an ideal tool for medical image-guided interventions. However, unlike computed tomography (CT) and magnetic resonance imaging (MRI) that provide innate three-dimensional (3D) anatomical models, ultrasound suffers from a lack of contextual correlations due to changing and unrecorded probe locations, which makes it challenging to be applied in certain clinical environments.

Existing methods for tracking ultrasound probes relative to human skin involve mounting cameras directly on the ultrasound probes. Such arrangements require specialized hardware and calibration within an operating room. Because the ultrasound probe must be in contact with the skin, previous algorithms have relied on the camera being at a fixed distance from the skin. Such methods do not allow the camera looking at the skin to be moved separately from the ultrasound probe or to be used both near and far from the skin.

A challenge for ultrasound in clinical applications stems from lacking a stable, anatomic coordinate system. One approach uses a feature tracking Scale-Invariant Feature Transform (SIFT) on images taken by a low-cost camera mounted on an ultrasound probe, and simultaneous localization and mapping (SLAM) for 3D reconstruction. While this method is cost effective, SIFT often fails to track natural skin features, resulting in high cumulative error. Other methods involve manually attaching known markers on the body. However, tracking tissue deformation requires a dense set of tracked points, and attaching or inking large numbers of artificial markers on a patient is not desirable. For example, many artificial markers protrude or cover the skin in a manner than can get in the way of the clinician, and artificial markers do not usually persist across months or years as would be desirable for longitudinal patient monitoring. Another method uses a commercial clinical 3D scanning system to acquire a preoperative 3D patient model, which aids in determining the location and orientation of the probe and the patient, but that method also mounted a camera directly on the ultrasound probe and was not usable with a mobile camera, such as a smartphone camera or head-mounted display (HMD). Another method uses phase only correlation (POC) tracking to robustly find subtle features with sub-pixel precision on the human body, but this POC method uses a camera mounted on the probe and is unable to track features when the camera is moved toward or away from the patient due to scale and rotation.

### SUMMARY

According to non-limiting embodiments or aspects, provided is a method for determining the pose of an object relative to a subject, comprising: capturing, with at least one computing device, a sequence of images with a stationary or movable camera unit arranged in a room, the sequence of images comprising a subject and an object moving relative to the subject; and determining, with at least one computing device, the pose of the object with respect to the subject in at least one image of the sequence of images based on a computing or using a prior surface model of the subject, a surface model of the object, and an optical model of the camera unit. In non-limiting embodiments or aspects, the at least one computing device and the camera unit are arranged in a mobile device. In non-limiting embodiments or aspects, the object being tracked may be at least one camera unit itself or at least one object physically connected to at least one camera unit. In non-limiting embodiments or aspects, the subject may be a medical patient. In other non-limiting embodiments or aspects, the subject may not be a patient. In non-limiting embodiments or aspects, the object(s) may be tracked for non-medical purposes, including but not limited to utilitarian or entertainment purposes. In other non-limiting embodiments or aspects, an animal or other subject with skin-like features may take the place of the subject.

In non-limiting embodiments or aspects, determining the pose of the object includes determining the skin deformation of the subject. In non-limiting embodiments or aspects, determining the pose of the object comprises: generating a projection of the surface model of the subject through the optical model of the camera unit; and matching the at least one image to the projection.

According to non-limiting embodiments or aspects, provided is a system for determining the pose of an object relative to a subject, comprising: a camera unit; a data storage device comprising a surface model of a subject, a surface model of an object, and an optical model of the camera unit; and at least one computing device programmed or configured to: capture a sequence of images with the camera unit while the camera unit is stationary and arranged in a room, the sequence of images comprising the subject and the object moving relative to the subject; and determine the pose of the object with respect to the subject in at least one image of the sequence of images based on a surface model of the subject, a surface model of the object, and an optical model of the camera unit.

In non-limiting embodiments or aspects, the at least one computing device and the camera unit are arranged in a mobile device. In non-limiting embodiments or aspects, wherein determining the pose of the object includes determining the skin deformation of the subject. In non-limiting embodiments or aspects, wherein determining the pose of the object comprises: generating a projection of the surface model of the subject through the optical model of the camera unit; and matching the at least one image to the projection.

According to non-limiting embodiments or aspects, provided is a system for determining the pose of an object relative to a subject, the system comprising: a camera not attached to the object able to view the object and the surface of the subject; a computer containing 3D surface models of the subject and the object, and an optical model of the camera; wherein: the computer determines the optimal 3D camera pose relative to the surface model of the subject for which the camera image of the subject best matches the surface model of the subject projected through the optical model of the camera; the computer uses the camera pose thus determined to find the optimal 3D object pose relative to the subject for which the camera image of the object best matches the surface model of the object projected through the optical model of the camera. In non-limiting embodiments or aspects, the camera is in a smartphone or tablet. In non-limiting embodiments or aspects, the object is a surgical tool. In other non-limiting embodiments or aspects, the camera is head mounted, including a camera incorporated into a head-mounted display.

In non-limiting embodiments or aspects, the object is an ultrasound probe. In non-limiting embodiments or aspects, the object is a clinician's hand or finger. In non-limiting embodiments or aspects, at least one of the surface model of the subject and the surface model of the object are derived from a set of images from a multi-camera system. In non-limiting embodiments or aspects, wherein at least one of the surface model of the subject and the surface model of the object are derived from a temporal sequence of camera images. In non-limiting embodiments or aspects, the optical model of the camera is derived from a calibration of the camera prior to the run-time operation of the system. In non-limiting embodiments or aspects, the optical model of the camera is derived during the run-time operation of the system.

In non-limiting embodiments or aspects, an inertial navigation system is incorporated into the object to provide additional information about object pose. In non-limiting embodiments or aspects, an inertial navigation system is incorporated into the camera to provide additional information about camera pose. In non-limiting embodiments or aspects, the inertial navigation system provides orientation and the video image provides translation for the camera pose. In non-limiting embodiments or aspects, inverse rendering of one or both of the surface models is used to find its optimal 3D pose. In non-limiting embodiments or aspects, a means is provided to guide the operator to move the object to a desired pose relative to the subject. In non-limiting embodiments or aspects, the operator is guided to move the object to an identical pose relative to the subject as was determined at a previous time. In non-limiting embodiments or aspects, the means to guide the operator makes use of the real-time determination of the present object pose. In non-limiting embodiments or aspects, the means to guide the operator identifies when a desired pose has been accomplished. In non-limiting embodiments or aspects, the operator is guided to move the object by selective activation of lights attached to the object. In non-limiting embodiments or aspects, the operator is guided to move the object by audio cues. In non-limiting embodiments or aspects, the operator is guided to move the object by tactile cues. In non-limiting embodiments or aspects, the operator is guided to move the object by a graphical display. In non-limiting embodiments or aspects, the graphical display contains a rendering of the object in the desired pose relative to the subject. In non-limiting embodiments or aspects, the object is virtual, comprising a single target point on the surface of the subject. In non-limiting embodiments or aspects, the object is virtual, comprising a one-dimensional line intersecting the surface of the subject at a single target point in a particular direction relative to the surface.

Further embodiments or aspects are set forth in the following numbered clauses:

Clause 1: A system for determining a pose of an object relative to a subject with a skin or skin-like surface, the system comprising: a camera not attached to the object and arranged to view the object and a surface of the subject; and a computing device in communication with the camera and comprising a three-dimensional (3D) surface model of the subject, a 3D surface model of the object, and an optical model of the camera, the computing device configured to: determine an optimal 3D camera pose relative to the 3D surface model of the subject for which an image of the subject captured by the camera matches the 3D surface model of the subject projected through the optical model of the camera; and determine an optimal 3D object pose relative to the subject for which an image of the object matches the 3D surface model of the object projected through the optical model of the camera.

Clause 2: The system of clause 1, wherein the camera is arranged in a smartphone or tablet.

Clause 3: The system of clauses 1 or 2, wherein the object is at least one of the following: a surgical tool, an ultrasound probe, a clinician's hand or finger, or any combination thereof.

Clause 4: The system of any of clauses 1-3, wherein at least one of the 3D surface models of the subject and the 3D surface models of the object is derived from a set of images from a multi-camera system.

Clause 5: The system of any of clauses 1-4, wherein at least one of the 3D surface models of the subject and the 3D surface models of the object is derived from a temporal sequence of camera images.

Clause 6: The system of any of clauses 1-5, wherein the optical model of the camera is derived from a calibration of the camera prior to a run-time operation of the system.

Clause 7: The system of any of clauses 1-6, wherein the optical model of the camera is derived during a run-time operation of the system.

Clause 8: The system of any of clauses 1-7, further comprising an inertial navigation system incorporated into the object and configured to output data associated with the optimal 3D object pose.

Clause 9: The system of any of clauses 1-8, further comprising an inertial navigation system incorporated into the camera and configured to output data associated with the optimal 3D camera object pose.

Clause 10: The system of any of clauses 1-9, wherein the inertial navigation system provides orientation data and a video image provides translation for the optimal 3D camera object pose.

Clause 11: The system of any of clauses 1-10, wherein determining at least one of the optimal 3D camera object pose and the optimal 3D object pose is based on an inverse rendering of at least one of the 3D surface model of the subject and the 3D surface model of the object.

Clause 12: The system of any of clauses 1-11, further comprising a guide configured to guide an operator to move the object to a desired pose relative to the subject.

Clause 13: The system of any of clauses 1-12, wherein the operator is guided to move the object to an identical pose relative to the subject that was determined at a previous time.

Clause 14: The system of any of clauses 1-13, wherein the guide is configured to guide the operator based on a real-time determination of a present object pose.

Clause 15: The system of any of clauses 1-14, wherein the guide identifies to the operator when a desired pose has been accomplished.

Clause 16: The system of any of clauses 1-15, further comprising lights attached to the object, wherein the operator is guided to move the object by selective activation of the lights.

Clause 17: The system of any of clauses 1-16, wherein the guide is configured to guide the operator based on audio cues.

Clause 18: The system of any of clauses 1-17, wherein the guide is configured to guide the operator based on tactile cues.

Clause 19: The system of any of clauses 1-18, wherein the guide is displayed on a graphical display.

Clause 20: The system of any of clauses 1-19, wherein the graphical display comprises a rendering of the object in the desired pose relative to the subject.

Clause 21: The system of any of clauses 1-20, wherein the object is a virtual object comprising a single target point on the surface of the subject.

Clause 22: The system of any of clauses 1-21, wherein the object is a virtual object comprising a one-dimensional line intersecting the surface of the subject at a single target point in a particular direction relative to the surface.

Clause 23: A method for determining a pose of an object relative to a subject, comprising: capturing, with at least one computing device, a sequence of images with a stationary or movable camera unit arranged in a room, the sequence of images comprising the subject and an object moving relative to the subject; and determining, with at least one computing device, the pose of the object with respect to the subject in at least one image of the sequence of images based on computing or using a prior surface model of the subject, a surface model of the object, and an optical model of the stationary or movable camera unit.

Clause 24: The method of clause 23, wherein the at least one computing device and the stationary or movable camera unit are arranged in a mobile device.

Clause 25: The method of clauses 23 or 24, wherein determining the pose of the object includes determining a skin deformation of the subject.

Clause 26: The method of any of clauses 23-25, wherein determining the pose of the object comprises: generating a projection of the surface model of the subject through the optical model of the stationary or movable camera unit; and matching at least one image to the projection.

Clause 27: A system for determining a pose of an object relative to a subject, comprising: a camera unit; a data storage device comprising a surface model of a subject, a surface model of an object, and an optical model of the camera unit; and at least one computing device programmed or configured to: capture a sequence of images with the camera unit while the camera unit is stationary and arranged in a room, the sequence of images comprising the subject and the object moving relative to the subject; and determine the pose of the object with respect to the subject in at least one image of the sequence of images based on a surface model of the subject, a surface model of the object, and an optical model of the camera unit.

Clause 28: The system of clause 27, wherein the at least one computing device and the camera unit are arranged in a mobile device.

Clause 29: The system of clauses 27 or 28, wherein determining the pose of the object includes determining skin deformation of the subject.

Clause 30: The system of any of clauses 27-29, wherein determining the pose of the object comprises: generating a projection of the surface model of the subject through the optical model of the camera unit; and matching the at least one image to the projection.

Clause 31: The method of any of clauses 27-30, wherein the object comprises the stationary or movable camera unit or at least one object physically connected to the stationary or movable camera unit.

Clause 32: A computer program product comprising at least one non-transitory computer-readable medium including program instructions that, when executed by at least one processor, cause the at least one processor to perform the methods of any of clauses 23-26 and 31.

Clause 33: The system of any of clauses 1-22 and 27-30, wherein the subject is a medical patient.

Clause 34: The method of any of clauses 23-26, wherein determining the pose of the object comprises tracking a feature on a skin surface of the subject by: identifying an image patch including the feature of an image from the sequence of images; building an image pyramid based on the image patch, the image pyramid comprising scaled versions of the image patch; and matching an image patch from a next image from the sequence of images to an image patch from the image pyramid.

Clause 35: The system of any of clauses 27-30, wherein the at least one computing device is programmed or configured to determine the pose of the object by tracking a feature on a skin surface of the subject by: identifying an image patch including the feature of an image from the sequence of images; building an image pyramid based on the image patch, the image pyramid comprising scaled versions of the image patch; and matching an image patch from a next image from the sequence of images to an image patch from the image pyramid.

Clause 36: A method for tracking a feature on a skin surface of a subject, comprising: detecting, with at least one computing device, feature points on an image of a sequence of images captured of the skin surface of the subject; identifying, with the at least one computing device, an image patch of the image including at least one feature point; building, with the at least one computing device, an image pyramid based on the image patch, the image pyramid comprising scaled versions of the image patch; matching, with the at least one computing device, an image patch from a next image from the sequence of images to an image patch from the image pyramid; and calculating, with the at least one computing device, a shift value for the next image based on matching the image patch from the next image to the image patch from the image pyramid.

Clause 37: The method of clause 36, further comprising: transforming the image patch of the image into a mathematical function; and extracting phase information from the image patch of the image, wherein matching the image patch is based on the phase information.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Additional advantages and details are explained in greater detail below with reference to the non-limiting, exemplary embodiments that are illustrated in the accompanying drawings, in which:

FIG. 1 illustrates a system for determining the pose of an object relative to a subject according to non-limiting embodiments;

FIG. 2 illustrates example components of a computing device used in connection with non-limiting embodiments;

FIG. 3 illustrates a flow chart for a method for determining the pose of an object relative to a subject according to non-limiting embodiments;

FIG. 4 illustrates a system for tracking features according to a non-limiting embodiment; and

FIG. 5 illustrates a flow chart for a method for tracking features according to non-limiting embodiments.

### DETAILED DESCRIPTION

It is to be understood that the embodiments may assume various alternative variations and step sequences, except where expressly specified to the contrary. It is also to be understood that the specific devices and processes described in the following specification, are simply exemplary embodiments or aspects of the disclosure. Hence, specific dimensions and other physical characteristics related to the embodiments or aspects disclosed herein are not to be considered as limiting. No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Also, as used herein, the terms “has,” “have,” “having,” or the like are intended to be open-ended terms. Further, the phrase “based on” is intended to mean “based at least partially on” unless explicitly stated otherwise.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. A computing device may also be a desktop computer or other form of non-mobile computer. In non-limiting embodiments, a computing device may include an artificial intelligence (AI) accelerator, including an application-specific integrated circuit (ASIC) neural engine such as Apple's M1® “Neural Engine” or Google's TENSORFLOW® processing unit. In non-limiting embodiments, a computing device may be comprised of a plurality of individual circuits.

As used herein, the term “subject” may refer to a person (e.g., a human body), an animal, a medical patient, and/or the like. A subject may have a skin or skin-like surface.

Non-limiting embodiments described herein utilize a camera detached and separate from an ultrasound probe or other object (e.g., clinical tool) to track the probe (or other object) by analyzing a sequence of images (e.g., frames of video) captured of a subject's skin and the features thereon. The camera may be part of a mobile device, as an example, mounted in a region or held by a user. The camera may be part of a head-mounted device (HMD). Although non-limiting embodiments are described herein with respect to ultrasound probes, it will be appreciated that such non-limiting embodiments may be implemented to track the position of any object relative to a subject based on the subject's skin features (e.g., blemishes, spots, wrinkles, deformations, and/or other parameters). For example, non-limiting embodiments may track the position of a clinical tool such as a scalpel, needle, a clinician's hand or finger, and/or the like.

Non-limiting embodiments may be implemented with a smartphone, using both the camera unit of the smartphone and the internal processing capabilities of the smartphone. A graphical user interface of the smartphone may direct a clinician, based on the tracked object, to move the object to a desired pose (e.g., position, orientation, and/or location with respect to the subject) based on a target or to avoid a critical structure, to repeat a previously-used pose, and/or to train an individual how to utilize a tool such as an ultrasound probe. In such non-limiting embodiments, no special instrumentation is needed other than a smartphone with a built-in camera and a software application installed. Other mobile devices may also be used, such as tablet computers, laptop computers, and/or any other mobile device including a camera.

In non-limiting embodiments, a data storage device stores three-dimensional (3D) surface models of a subject (e.g., patient) and an object (e.g., ultrasound probe). The data storage device may also store an optical model of a camera unit. The data storage device may be internal to the computing device or, in other non-limiting embodiments, external to and in communication with the computing device over a network. The computing device may determine a closest match between the subject depicted in an image from the camera and the surface model of the subject projected through the optical model of the camera. The computing device may determine an optimal pose of the camera unit relative to the surface model of the subject. The computing device may determine a closest match between the object depicted in the image and the surface model of the object projected through the optical model of the camera. The computing device may determine an optimal pose for the object relative to the subject.

Non-limiting embodiments provide for skin-feature tracking usable in an anatomic simultaneous localization and mapping (SLAM) algorithm and localization of clinical tools relative to the subject's body. By tracking features from small patches of a subject's skin, the unique and deformable nature of the skin is contrasted to objects (such as medical tools and components thereof) that are typically rigid and can be tracked with 3D geometry, allowing for accurate tracking of objects relative to a subject in real-time. The use of feature tracking in a video taken by a camera (e.g., such as a camera in a smartphone or tablet) and anatomic SLAM of the camera motion relative to the skin surface allows for accurate and computationally efficient camera-based tracking of clinical tool(s) relative to reconstructed 3D features from the subject. In non-limiting examples, the systems and methods described herein may be used for freehand smartphone-camera based tracking of natural skin features relative to tools. In some examples, robust performance may be achieved with the use of a phase-only correlation (POC) modified to uniquely fit the freehand tracking scenario, where the distance between the camera and the subject varies over time.

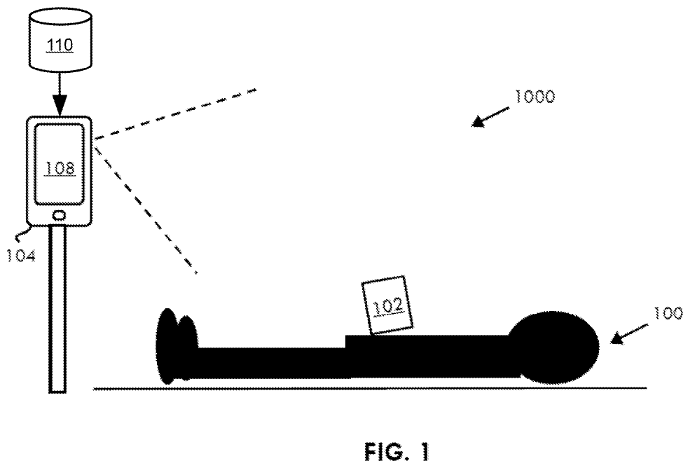

FIG. 1 shows a system 1000 for determining the pose of an object 102 relative to a subject 100 (e.g., person or animal) according to a non-limiting embodiment. The system 1000 includes a computing device 104, such as a mobile device, arranged in proximity to the subject 100. A clinician or other user (not shown in FIG. 1) manipulates the object 102, which may be an ultrasound probe or any other type of object, with respect to the subject 100. The computing device 104 includes a camera having a field of view that includes the object 102 and the subject 100. In some examples, the camera may be separate from the computing device 104. The computing device 104 may be mounted in a stationary manner as shown in FIG. 1, although it will be appreciated that a computing device 104 and/or camera may also be held by a user in non-limiting embodiments.

In non-limiting embodiments, the computing device 104 also includes a graphical user interface (GUI) 108 which may visually direct the user of the computing device 104 to guide the object 102 to a particular pose. For example, a visual guide may be generated on the GUI 108 to direct a clinician to a particular area of the skin surface. The computing device 104 may also guide the user with audio. The system 1000 includes a data storage device 110 that may be internal or external to the computing device 104 and includes, for example, an optical model of the camera, a 3D model of the subject and/or subject's skin surface, a 3D model of the object (e.g., clinical tool) to be used, and/or other like data used by the system 1000. The 3D models of the subject, subject's skin surface, and/or object may be represented in various ways, including but not limited to 3D point clouds.

In non-limiting embodiments, natural skin features may be tracked using POC to enable accurate 3D ultrasound tracking relative to skin. The system 1000 may allow accurate two-dimensional (2D) and 3D tracking of natural skin features from the perspective of a free-hand-held smartphone camera, including captured video that includes uncontrolled hand motion, distant view of skin features (few pixels per feature), lower overall image quality from small smartphone cameras, and/or the like. The system 1000 may enable reliable feature tracking across a range of camera distances and working around physical limitations of smartphone cameras.

In non-limiting embodiments, at least one camera unit may be attached to an HMD. In some examples, the HMD may contain an augmented-reality or virtual-reality display that shows objects inside or relative to the subject's skin, such that the objects appear to move with the skin. In non-limiting embodiments or aspects, the HMD may show medical images or drawings at their correct location in-situ inside the subject's body, such that the images move with the subject's skin. In non-limiting embodiments or aspects, a camera attached to an HMD may simultaneously track an ultrasound probe along with the subject's skin, and the HMD could show the operator current and/or previous images (and/or content derived from the images) in their correct location inside the subject's body (or at any desired location in 3D space that moves with the subject's skin on the subject's body), whether the subject's body remains still, moves, or is deformed.

In non-limiting embodiments, the object 102 may be a virtual object including a one-dimensional (1D) line intersecting the surface of the subject at a single target point in a particular direction relative to the surface.

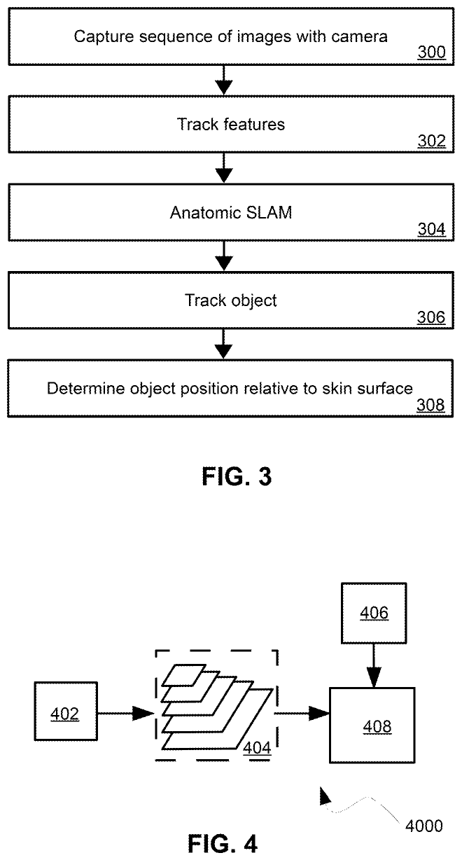

Referring now to FIG. 3, a flow diagram is shown for a method of determining the pose of an object relative to a subject according to non-limiting embodiments. The steps shown in FIG. 3 are for example purposes only. It will be appreciated that additional, fewer, different, and/or a different order of steps may be used in non-limiting embodiments. At a first step 300, a sequence of images are captured with a camera. For example, a video camera of a mobile device (e.g., such as a smartphone with an integrated camera, an HMD, and/or the like) may be used to capture a sequence of images (e.g., frames of video) that include a subject's skin surface and at least one object (such as an ultrasound probe or other tool).

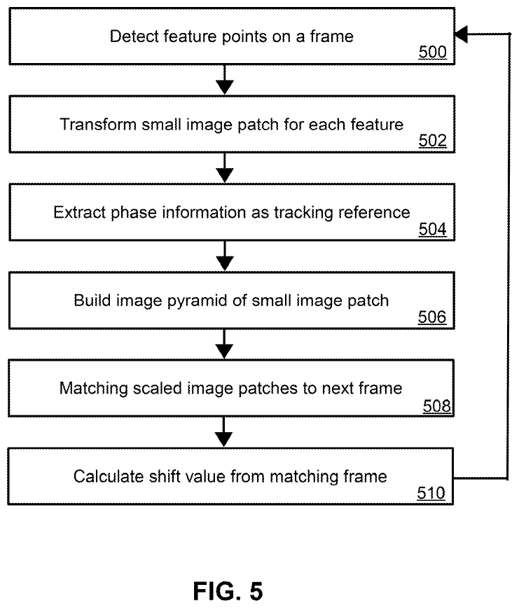

At step 302 of FIG. 3, the features from the subject's skin are tracked using POC. Non-limiting examples of feature tracking are described herein with respect to FIGS. 4 and 5. At step 304, which may be performed simultaneously or substantially simultaneously with step 302, an anatomic SLAM algorithm is applied to construct a mapping of the subject's 3D skin surface and the features thereon with respect to the motion of the camera. In non-limiting embodiments, a visual SLAM approach may be implemented by defining a set S containing five (5) consecutive captured frames f_{i,j}, Sᵢ=f_{i,0}, f_{i,1}, f_{i,2}, f_{i,3}, f_{i,4}. Other numbers of frames may be used. In f_{i,0}, a GFtT process is used to find initial features with a constraint that the features are at least a predetermined number of pixels (e.g., five (5) pixels) away from each other because POC requires separation between features to operate reliably. At step 302, from f_{i,1} to f_{i,4}, the scale-invariant tracking system and method shown in FIGS. 4 and 5 may be used to track the corresponding features along the remainder of the frames. After tracking features from the set Sᵢ, the tracked acceptable features of f_{i,4} will be inherited by f_{i+i,0} in the new set Sᵢ₊₁. After setting f_{i+1,0}=f_{i,4}, the process shown in steps 300-302 are repeated for Sᵢ₊₁ by finding new features from the areas of f_{i+1,0} that lack features, while the inherited features provide an overlap to maintain correspondence between the two (2) sets. A mask (e.g., an 11×11 mask) may be applied on top of every inherited feature to avoid finding features that are too close to each other. Feature tracking is again performed in Sᵢ₊₁ and the process continues until the end of the sequence of images.

In non-limiting embodiments, prior to capturing the sequence of images at step 300 with the camera, an optical model may be generated for the camera through which other data may be projected. An optical model may be based on an intrinsic matrix obtained by calibrating the camera. As an example, several images of a checkerboard may be captured from different viewpoints and feature points may be detected on the checkerboard corners. The prior known position of the corners may then be used to estimate the camera intrinsic parameters, such as a focal length, camera center, and distortion coefficient, as examples. In some examples, the camera calibration toolbox provided by Matlab may be utilized. In non-limiting embodiments, the optical model may be predefined for a camera. The resulting optical model may include a data structure including the camera parameters and may be kept static during the tracking process. In some examples, to prepare for tracking, preprocessing may be performed to convert color images to grayscale and to then enhance the appearance of skin features using contrast limited adaptive histogram equalization (CLAHE) to find better spatial frequency components for the feature detection and tracking.

While processing the sequence of images, a Structure from Motion (SfM) process may be performed locally on every newly obtained set Sᵢ, and the locally computed 3D positions may be used to initialize the global set S={S₀, S₁, S₂}. Once a new set is obtained and added to global set, a Bundle Adjustment process may be used to refine the overall 3D scheme. Through this process, it is possible to simultaneously update and refine the 3D feature points and camera motions while reading in new frames from the camera. In non-limiting embodiments, rather than using an existing SfM process, which includes a normalized five-point algorithm and random sample consensus, a modified process is performed to minimize re-projection error in order to compute structure from motion for every several (e.g., five (5)) frames. First, re-projection error is defined by the Euclidean distance ∥x−xᵣₑₚ∥², where x is a tracked feature point and xᵣₑₚ is a point obtained by projecting a 3D point back to the image using the calculated projection matrix (e.g., optical model) of the camera. After obtaining the initialized 3D points, camera projection matrix (including Intrinsic Matrix and Extrinsic Matrix), and corresponding 2D features in a set, the re-projection error is minimized. In this latter stage, the Intrinsic Matrix is fixed and the system updates the 3D points and camera Extrinsic Matrix repeatedly.

For higher robustness, an additional constraint may be set in some non-limiting embodiments that new feature points must persist across at least two (2) consecutive sets before they are added to the 3D model (e.g., point cloud) of the subject. Higher reconstruction quality may be achieved by setting larger constraint thresholds. Due to the use of SfM, the resulting 3D model of the arm and the camera trajectory are only recovered up to a scale factor. In some examples, the 3D positions may be adjusted to fit into real-world coordinates. In some examples, a calibrated object (such as a ruler or a small, flat fiducial marker (e.g., an AprilTag)) may be placed on the subject's skin during a first set of frames of the video.

With continued reference to FIG. 3, at step 306 the object is tracked relative to 3D reconstructed features from the subject's 3D skin surface. Step 306 may be performed simultaneously with steps 302 and 304. At step 308 the position of the object is determined relative to the subject's 3D skin surface. This may involve localizing the object in the environment and may facilitate, for example, a system to automatically guide an operator to move the object and/or perform some other task with respect to the subject's skin surface.

In non-limiting embodiments, fiducial markers (e.g., such as an Apriltag) may be placed on objects (e.g., such as clinical tools). In this manner, the fiducial marker(s) may be used to accurately track the 3D position of the object during use. The fiduciary markers may also be masked while tracking skin surface features. After reconstructing the 3D skin surface during a first portion of the video, as described herein, one or more objects may be introduced. The computing device may continue to execute SfM and Bundle Adjustment algorithms while the object moves with respect to the skin surface (e.g., such as a moving ultrasound probe) to accommodate the hand-held movement of the camera and possible skin deformation or subject motion. Continuous tracking of both the skin features and objects relative to the moving camera allows consistent tracking of the objects relative to the skin features. In some examples, this feature tracking approach may also find POC features on objects, which may confuse 3D reconstruction of the skin surface. This problem is addressed by first detecting the fiduciary marker on the object and then masking-out the object from the images (e.g., based on a known geometry) before performing feature detection.

FIG. 4 illustrates a system 4000 for scale-invariant feature tracking according to non-limiting embodiments. The system 4000 may be a subsystem and/or component of the system 1000 shown in FIG. 1. An image patch 402 may be a small image patch centered on a feature of the subject's skin surface. This may be performed with small image patches centered on each feature of a plurality of features. This image patch 402 is used to generate an image pyramid 404, which includes several different scales of the image patch 402. The different scales in the image pyramid 404 are used by a computing device 408 to match to a corresponding image patch 406 from a next frame in the sequence of captured images. In this manner, if the distance between the camera and the skin surface changes between frames, a scaled image from the image pyramid 404 enables for accurate matching and continued tracking of the features.

Referring now to FIG. 5, and with continued reference to FIG. 4, a flow diagram is shown for a method of scale-invariant feature tracking according to non-limiting embodiments. The steps shown in FIG. 5 are for example purposes only. It will be appreciated that additional, fewer, different, and/or a different order of steps may be used. At a first step 500, feature points are detected in a first frame. As an example, this may use a Good Features to Track (GFtT) method or any other like method to detect the features. Steps 502-510 represent a modified POC approach. At step 502, small image patches (e.g., image patch 402) centered on each feature identified at step 500 are transformed into a mathematical function. For example, a Fourier Transformation may be performed on each image patch.

At step 504 of FIG. 5, phase information is extracted from each transformed image patch (e.g., image patch 402) to be used as a tracking reference (e.g., to be used to match to a next frame). At step 506, rather than directly proceeding to matching the transformed image patch (e.g., image patch 402) to a corresponding image patch (e.g., image patch 406) in a following frame, the method involves building an image pyramid 404 based on the image patch 402 in the frame being processed (e.g., the target frame) to accommodate a possible change in scale that results from a change in distance between the camera and the skin surface. The image pyramid 404 is built by generating a number of scaled versions of the image patch 402 at different scales. At step 508, each of the images in the image pyramid 404 are used to match against a corresponding image patch 406 in a next frame by determining a similarity score and determining if the similarity score satisfies a confidence threshold (e.g., meets or exceeds a predetermined threshold value). In some examples, similarity scores may be generated for each image in the image pyramid 404 and the highest scoring image patch may be identified as a match. At step 508, if an image patch becomes too sparse with too few tracked feature points, the method may proceed back to step 500 to identify new features in the sparse region and such process may be repeated so that the sequence of images includes an acceptable number of well-distributed tracked feature points.

At step 510 of FIG. 5, a shift value is calculated based on the matching image patch to shift the spatial position of the next frame (e.g., the frame including image patch 406) for continued, accurate tracking. The shift value may provide 2D translation displacements with sub-pixel accuracy. The method then proceeds back to step 500 to process the next frame, using the currently-shifted frame as the new target frame and the following frame to match. Through use of the image pyramid 404, the number of feature points that are eliminated during tracking is reduced from existing tracking methods that often result in the rapid loss of feature points during tracking when the camera is displaced. Non-limiting embodiments of a scale-invariant feature tracking process as shown in FIG. 5 track at least as many feature points as methods not using an image pyramid (because the image pyramid includes the original scale patch) as well as additional, more effective (e.g., for more accurate and efficient tracking) feature points that are not obtained by other methods.

In non-limiting embodiments, a wide field of view of the camera may introduce spurious objects that should not be tracked (e.g., an additional appendage, tool, or the like). This may be addressed in non-limiting embodiments by automatically identifying which pixels correspond to the subject using human pose tracking and semantic segmentation, as an example. In non-limiting embodiments, a color background (e.g., a blue screen) may be used to isolate the subject's skin by masking the color image. After masking the color image, the image may be converted to grayscale for further processing. In non-limiting embodiments, masks may be applied to mask known objects (e.g., an ultrasound probe).

In non-limiting embodiments, motion blur may be reduced or eliminated by forcing a short shutter speed. For example, the camera may be configured to operate at 120 frames per second (fps), of which every 20th frame (or other interval) is preserved to end up at a target frame rate (e.g., 6 fps). The target frame rate may be desirable because SfM requires some degree of motion within each of the sets Sᵢ, which is achieved by setting a lower frame rate resulting in a 0.8 second duration for each Sᵢ. The 3D model reconstruction may be updated every several (e.g., four (4)) captured frames (with 6 fps, the fifth and first frames of consecutive sets overlap), and the 3D skin feature tracking may be updated every two-thirds second, as an example. In non-limiting embodiments, rotational invariance may be integrated into the POC tracking of skin features.

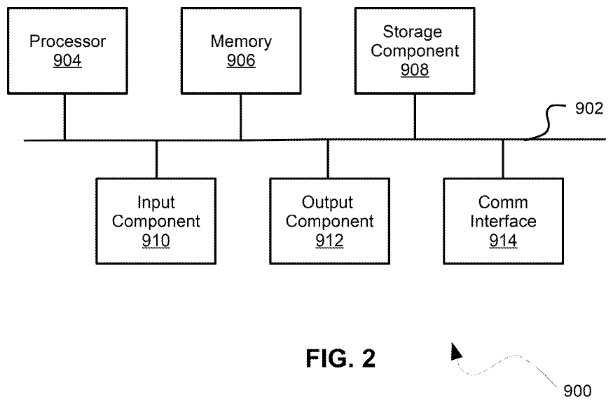

Referring now to FIG. 2, shown is a diagram of example components of a computing device 900 for implementing and performing the systems and methods described herein according to non-limiting embodiments. In some non-limiting embodiments, device 900 may include additional components, fewer components, different components, or differently arranged components than those shown in FIG. 2. Device 900 may include a bus 902, a processor 904, memory 906, a storage component 908, an input component 910, an output component 912, and a communication interface 914. Bus 902 may include a component that permits communication among the components of device 900. In some non-limiting embodiments, processor 904 may be implemented in hardware, firmware, or a combination of hardware and software. For example, processor 904 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 906 may include random access memory (RAM), read only memory (ROM), and/or another type of dynamic or static storage device (e.g., flash memory, magnetic memory, optical memory, etc.) that stores information and/or instructions for use by processor 904.

With continued reference to FIG. 2, storage component 908 may store information and/or software related to the operation and use of device 900. For example, storage component 908 may include a hard disk (e.g., a magnetic disk, an optical disk, a magneto-optic disk, a solid state disk, etc.) and/or another type of computer-readable medium. Input component 910 may include a component that permits device 900 to receive information, such as via user input (e.g., a touch screen display, a keyboard, a keypad, a mouse, a button, a switch, a microphone, etc.). Additionally, or alternatively, input component 910 may include a sensor for sensing information (e.g., a global positioning system (GPS) component, an accelerometer, a gyroscope, an actuator, etc.). Output component 912 may include a component that provides output information from device 900 (e.g., a display, a speaker, one or more light-emitting diodes (LEDs), etc.). Communication interface 914 may include a transceiver-like component (e.g., a transceiver, a separate receiver and transmitter, etc.) that enables device 900 to communicate with other devices, such as via a wired connection, a wireless connection, or a combination of wired and wireless connections. Communication interface 914 may permit device 900 to receive information from another device and/or provide information to another device. For example, communication interface 914 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 900 may perform one or more processes described herein. Device 900 may perform these processes based on processor 904 executing software instructions stored by a computer-readable medium, such as memory 906 and/or storage component 908. A computer-readable medium may include any non-transitory memory device. A memory device includes memory space located inside of a single physical storage device or memory space spread across multiple physical storage devices. Software instructions may be read into memory 906 and/or storage component 908 from another computer-readable medium or from another device via communication interface 914. When executed, software instructions stored in memory 906 and/or storage component 908 may cause processor 904 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “programmed or configured,” as used herein, refers to an arrangement of software, hardware circuitry, or any combination thereof on one or more devices.

Although embodiments have been described in detail for the purpose of illustration, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system for determining a pose of an object relative to a subject with a skin or skin-like surface, the system comprising:
a camera not attached to the object and arranged to view the object and a surface of the subject; and
a computing device in communication with the camera and comprising a three-dimensional (3D) surface model of the subject, a 3D surface model of the object, and an optical model of the camera, the computing device configured to:
determine an optimal 3D camera pose relative to the 3D surface model of the subject for which an image of the subject captured by the camera matches the 3D surface model of the subject projected through the optical model of the camera; and
determine an optimal 3D object pose relative to the subject for which an image of the object matches the 3D surface model of the object projected through the optical model of the camera.

2. The system of claim 1, wherein the camera is arranged in a smartphone or tablet.

3. The system of claim 1, wherein the object is at least one of the following: a surgical tool, an ultrasound probe, a clinician's hand or finger, or any combination thereof.

4. The system of claim 1, wherein at least one of the 3D surface models of the subject and the 3D surface models of the object is derived from a set of images from a multi-camera system.

5. The system of claim 1, wherein at least one of the 3D surface models of the subject and the 3D surface models of the object is derived from a temporal sequence of camera images.

6. The system of claim 1, wherein the optical model of the camera is derived from a calibration of the camera prior to a run-time operation of the system.

7. The system of claim 1, wherein the optical model of the camera is derived during a run-time operation of the system.

8. The system of claim 1, further comprising an inertial navigation system incorporated into the object and configured to: output data associated with at least one of the optimal 3D object pose and the optimal 3D cameral object pose.

9-10. (canceled)

11. The system of claim 1, wherein determining at least one of the optimal 3D camera object pose and the optimal 3D object pose is based on an inverse rendering of at least one of the 3D surface model of the subject and the 3D surface model of the object.

12. The system of claim 1, further comprising a guide configured to guide an operator to move the object to a desired pose relative to the subject.

13. (canceled)

14. The system of claim 12, wherein the guide is configured to guide the operator based on a real-time determination of a present object pose.

15. The system of claim 14, wherein the guide identifies to the operator when a desired pose has been accomplished.

16. The system of claim 12, further comprising lights attached to the object, wherein the operator is guided to move the object by selective activation of the lights.

17. The system of claim 12, wherein the guide is configured to guide the operator based at least one of audio cues and tactile cues.

18. (canceled)

19. The system of claim 12, wherein the guide is displayed on a graphical display comprises a rendering of the object in the desired pose relative to the subject.

20. (canceled)

21. The system of claim 1, wherein the object is a virtual object comprising a single target point on the surface of the subject.

22. The system of claim 1, wherein the object is a virtual object comprising a one-dimensional line intersecting the surface of the subject at a single target point in a particular direction relative to the surface.

23. A method for determining a pose of an object relative to a subject, comprising:
capturing, with at least one computing device, a sequence of images with a stationary or movable camera unit arranged in a room, the sequence of images comprising the subject and an object moving relative to the subject; and
determining, with at least one computing device, the pose of the object with respect to the subject in at least one image of the sequence of images based on computing or using a prior surface model of the subject, a surface model of the object, and an optical model of the stationary or movable camera unit.

24. The method of claim 23, wherein the at least one computing device and the stationary or movable camera unit are arranged in a mobile device.

25. The method of claim 23, wherein determining the pose of the object includes determining a skin deformation of the subject.

26. The method of claim 23, wherein determining the pose of the object comprises:
generating a projection of the surface model of the subject through the optical model of the stationary or movable camera unit; and
matching at least one image to the projection.

27. A system for determining a pose of an object relative to a subject, comprising:
a camera unit;
a data storage device comprising a surface model of a subject, a surface model of an object, and an optical model of the camera unit; and
at least one computing device programmed or configured to:
capture a sequence of images with the camera unit while the camera unit is stationary and arranged in a room, the sequence of images comprising the subject and the object moving relative to the subject; and
determine the pose of the object with respect to the subject in at least one image of the sequence of images based on a surface model of the subject, a surface model of the object, and an optical model of the camera unit.

28-37. (canceled)
